(19) United States
(12) Patent Application Publication
(10) Pub. No.: US 2021/0330852 A1
(43) Pub. Date: Oct. 28, 2021

(54) ULTRAVIOLET IRRADIANCE OPTIMIZATION CHAMBER

(71) Applicants: Sean Patrick Pawlicki, San Diego, CA (US); Huggy Lamar Price, Carlsbad, CA (US)

(72) Inventors: Sean Patrick Pawlicki, San Diego, CA (US); Huggy Lamar Price, Carlsbad, CA (US)

(73) Assignee: Sean Pawlicki pawlicki, San Diego, CA (US)

(21) Appl. No.: 17/234,788

(22) Filed: Apr. 19, 2021

Related U.S. Application Data
(60) Provisional application No. 63/012,008, filed on Apr. 17, 2020.

(51) Int. Cl. A61L 9/20; F24F 8/22

(52) U.S. Cl. CPC A61L 9/20; F24F 8/22; A62B 18/08; A61L 2209/16; A61L 2209/12

(57) ABSTRACT

A ultraviolet irradiance optimization chamber comprising a first opening, an inner chamber, a second opening, and a UVC source. The UVC source is preferably adjacent to the first opening, with sufficient room for a fluid to enter the chamber. The chamber is multifaceted with a plurality of angles of different degrees designed to act as a light channeling optic through the ricocheting of UVC radiation within the chamber to achieve homogeneity of UVC irradiation to the fluid within the chamber.

### CROSS-REFERENCE TO RELATED APPLICATIONS

This application claims the benefit of U.S. Provisional Application No. 63/035,916, filed Jun. 8, 2020, entitled “UVC Irradiance Optimization Chamber,” and U.S. Provisional Application No. 63/012,008, filed Apr. 17, 2020, entitled “UVC Irradiance Optimization Chamber,” the entireties of which are incorporated herein by reference.

### BACKGROUND OF THE INVENTION

### Field of the Invention

The present invention relates to novel UV sterilization chambers for disinfection of fluids. More particularly the invention relates to the dispersion of UV within a sterilization camber to produce a more uniform irradiance pattern.

### Description of the Related Art

UVC sterilization of fluids has been used to deactivate pathogens in the subject material. Ultraviolet germicidal irradiation (UVGI) is a disinfection method that uses short-wavelength ultraviolet (ultraviolet C or UVC) light to kill or inactivate microorganisms by destroying nucleic acids and disrupting their DNA, leaving them unable to perform vital cellular functions. UVGI is used in a variety of applications, such as food, air, and water purification.

The germicidal wavelengths exist in the UVC subsection of the UV spectrum. Ultraviolet (UV) Light has applications in the field of water treatment, optical data storage, communications, biological agent detection and polymer curing. The UVC region of the UV spectral range refers to wavelengths between 100 nm to 280 nm. In the case of disinfection, the optimum wavelength is in the region of 260 nm to 270 nm, with germicidal efficacy falling exponentially with longer wavelengths. With respect to the DNA of pathogens and their susceptibility to the UVC light, DNA, RNA, and proteins will absorb biological organisms exposed to deep UV light in the range of 200 nm to 300 nm. UVC photons penetrate cells and damage the nucleic acid, rendering them incapable of reproduction or microbiologically inactive. This same process occurs in nature when the sun emits UV rays. Absorption by proteins can lead to rupture of cell walls and death of the organism. Absorption by DNA or RNA, specifically by thymine bases, is known to cause inactivation of the DNA or RNA double helix strands through the formation of thymine dimers. If enough of these dimers are created in DNA, the DNA replication process is disrupted, and the cell cannot replicate. In an embodiment of the invention, the high-powered light is in the range of 265 nm-275 nm.

### BRIEF SUMMARY OF THE INVENTION

Embodiments of the present invention address deficiencies of the art in respect to irradiation within a chamber. The chamber may include any fluid or gas. In a preferred embodiment, a UVC chamber includes a top and a bottom opposite the top surface, where the top and the bottom are substantially flat and substantially parallel to each other. The UVC chamber further includes a first side and a second side opposite the first side, where the first side and the second side are substantially flat and substantially parallel to each other, and where the top, bottom, first side and second side form a substantially polygonal configuration. The UVC chamber further includes a proximal face and a distal face opposite the proximal face enclosing the top, bottom, first side and second side forming an interior and an exterior of the UVC chamber, where the distal face includes one or more substantially flat surfaces. The UVC chamber further includes a first opening adjacent the proximal face, the opening configured to allow the flow of fluid to the interior of the UVC chamber and a second opening configured to allow the flow of fluid to the exterior of the UVC chamber. The UVC chamber further includes a UVC light source positioned adjacent to the first opening of the UVC chamber and the interior of the UVC chamber includes a plurality of facets configured at different angles to maximize internal reflection of UVC within the UVC chamber and the interior of the UVC chamber further includes reflective material configured to maximize internal reflection of UVC within the UVC chamber.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 100 a diagram illustrating an improved UVC irradiation chamber configured to act as a secondary optic used to channel UVC radiation from an LED light source in accordance with an embodiment of the invention;

FIG. 200 is a diagram illustrating an improved irradiation chamber configured to act as a secondary optic used to channel UVC radiation from a mercury lamp light source in accordance with an embodiment of the invention;

FIG. 300 is diagram illustrating an improved irradiation chamber configured to use a secondary optic to further channel UVC radiation in accordance with an embodiment of the invention;

FIG. 400 is a diagram illustrating the use of the UVC irradiation chamber as a tertiary optic to further channel UVC radiation coming from in accordance with an embodiment of the invention;

### DETAILED DESCRIPTION OF THE INVENTION

The present disclosure comprises an improved irradiation chamber configured as a radiation channeling optic to maximize the efficacy of a UV light source thereby improving the irradiation of fluids passed through the chamber. The improved chamber comprises a first opening, a multifaceted chamber designed to internally reflect the UV light source within the chamber through the use of a combination of diffusive and/or diffractive structures, and a second opening. The fluid enters the first opening and exits through the second opening after having been treated by the UVC within the chamber. In a preferred embodiment, a UVC light source is positioned adjacent the first opening and used to flood the chamber with UVC. The chamber is configured with a plurality of facets configured at different angles in order to maximize the internal reflection of the UVC within the chamber.

This improved chamber design may be utilized in connection with any fluid treating system. For example, the improved chamber may be configured to be used with an HVAC system, or any other air treatment system. In alternative embodiments, the improved chamber may be carried and worn with a user to work with treating air used by the user, such as in the case of a mask.

A UVC irradiance optimization chamber in accordance with the present disclosure includes a first and second opening, and an internal chamber designed to maximize the efficacy of the UVC within the chamber. Preferably, a UVC source is installed adjacent to an opening. In a preferred embodiment, the UVC source may be configured adjacent an opening to provide sufficient room for the fluid to enter the chamber at the same location as the UVC enters the chamber. In a preferred embodiment, the chamber is multifaceted with a plurality of angles, wherein the angles are different from one another, to maximize the ricocheting of UVC light within the chamber. Through this ricocheting, the UVC is applied uniformly throughout the chamber, resulting in fluids or gasses being treated uniformly. Historical approaches to maximize the heterogeneity of UVC (or other source) within a chamber has required the addition of additional light sources. This presents issues in that additional light sources require additional power supplies. Through the advantages of this disclosure, UVC (or other light source) can be maximized within a chamber without need for adding additional light sources, resulting in lower power demands.

Another preferred embodiment further improves radiation homogeneity by incorporating and attaching a secondary optic to the first opening of the chamber. Said secondary optic will be comprised of a transparent material and will use a convex lens, a diffuser, or some combination of a convex lens or diffuser to more efficiently channel UVC radiation. In this preferred embodiment the irradiation chamber acts as a tertiary optic that disperses the light more evenly.

The complexity and configuration of the UVC chamber allows for the least amount of energy used to maximize the output power and achieve required dosage to disinfect the coronavirus and other pathogens. FIG. 400 illustrates the irradiance intensity of a chamber in accordance with the present disclosure.

Appropriate reflective and shielding materials are used to maximize the UVC throughout the chamber, as well as improve the efficacy throughout the chamber so that there is more consistent and uniform UVC energy throughout the chamber with fewer hotspots.

Embodiments of the invention provide for a mask with a UVC Chamber as an improvement to masks, respirators, face covers (“masks”) by using a UVC chamber in connection with the mask to sterilize the air breathed. In an embodiment of the invention, the UVC chamber is a UVC irradiance optimization chamber that is designed to maximize the efficacy of the UVC within the chamber to irradiate biological pathogens more efficiently. In order to maximize the efficacy of UVC in this embodiment of the invention, the UVC source is positioned at an opening to the UVC chamber to the where fluid or gas enters the chamber. The UVC source may be configured adjacent to the opening to provide sufficient room for the gas to enter the chamber at the same location as the UVC enters the chamber. In an embodiment of the invention, the chamber is multifaceted with a plurality of angles, and the angles may be different from one another, to maximize the ricocheting of UVC light within the chamber. Through this ricocheting, the UVC is applied uniformly throughout the chamber, resulting in gasses being treated uniformly. The gas or fluid then exits the chamber through another opening. In an embodiment of the invention, one of the openings corresponds to where end user is breathing through the mask and one of the openings corresponds to where the gas enters and exits from an external environment. Thus, the user is able to freely inhale air that has been irradiated of biological pathogens through UVC and when the user exhales, the air that is expelled is also irradiated of biological pathogens.

Further, as this chamber maximizes the impact of the UVC within the chamber to more uniformly apply the UVC to the fluid or gas to be treated, the chamber results in maximum UVC performance without need to increase the level of energy supplied. Because the UVC chamber is optimized to maximize the UVC light within the chamber, the irradiation chamber further enables the use of a UVGI cartridge in a smaller and more practical size that can be used with numerous options, including portable use and wearing in connection with a face mask, which would not otherwise be possible. Thus, the UVC chamber is entirely self-contained within the portable mask. Not only does this permit smaller chamber designs, the chamber allows for maximum efficiency in larger designs, such as any HVAC system or any two-way air purification system.

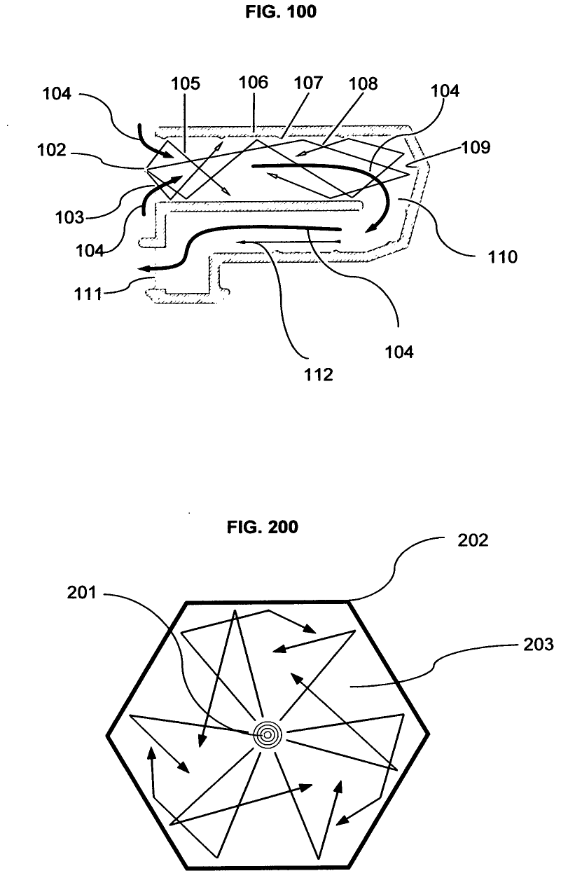

In further illustration, referring now to the drawings in which like numerals represent the same or similar elements and initially to FIG. 100, an irradiation chamber FIG. 100 is shown in accordance with one illustrative embodiment. The chamber 101 is comprised of materials that diffuse light, or are coated with a substance that diffuses light. The UVC LED 102 emits light at 150 degrees from its center of axis. The light is placed adjacent to the chamber's first opening 103 and, together with the fluid 104 medium, enters the chamber. The UVC light proceeds generally along multiple paths 105 until the light encounters a wall of the chamber 106. The wall contains multiple facets 107 and/or angles such that the light is reflected in multiple angles 109 around the chamber FIG. 100. The fluid enters at opening 103 then generally proceeds to the wall of the chamber 109 located opposite the opening 103. The fluid continues to flow through an interior path 110 and eventually exits the chamber at 111. As the fluid flows along interior pathway 110 it is irradiated through exposure to UVC radiation. In a preferred embodiment the irradiation chamber may be used with masks or respirators and provide a novel and non-obvious apparatus for a portable means of irradiating biological pathogens from air. In said embodiment the fluid (air) flows back along a reverse interior path 112. Such a configuration disinfects both inhaled and exhaled air, meaning contaminated droplets from infected victims or asymptomatic carriers are no longer a threat to others.

FIG. 200 shows an irradiation chamber FIG. 200 utilizing a mercury lamp 201 as the UVC light source as is customary practice. The lamp 201 typically runs the length of the chamber 202 with UVC rays 203 emitted at 90 degrees from the lamp. In a preferred embodiment of our chamber, the chamber 202 is shaped in such a way to reflect the UVC light away from the center of chamber to achieve irradiation homogeneity.

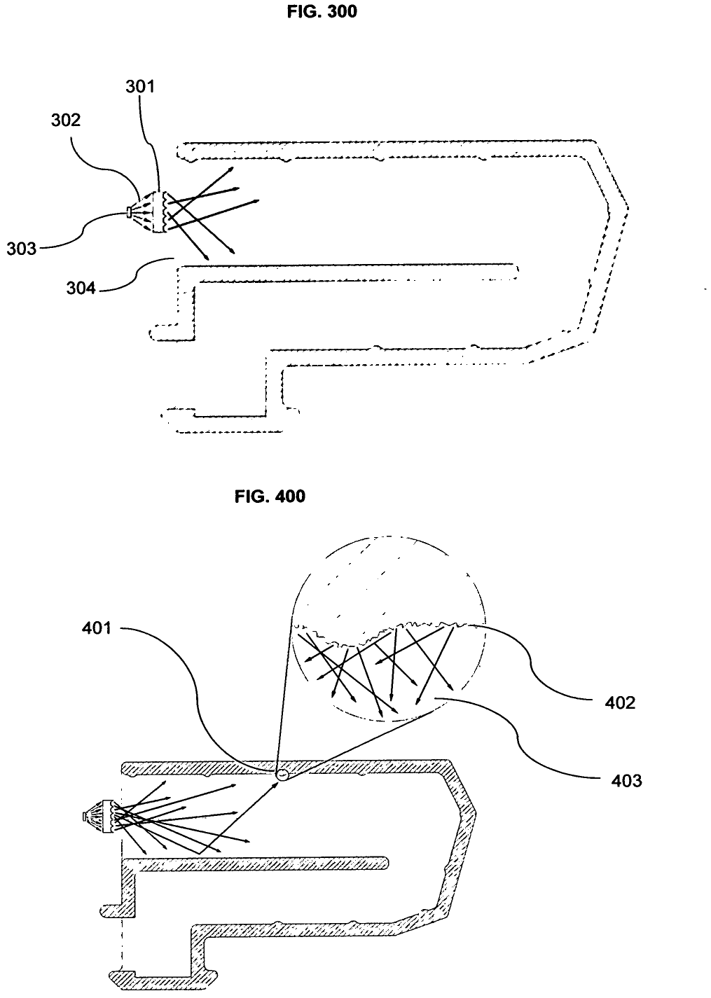

In FIG. 300 an improved irradiation chamber FIG. 300 is configured to use a secondary optic 301 comprising a transparent material using a convex or concave lens or other diffusive angular combinations, a diffuser, or some combination of a convex lens or diffuser. Said secondary optic intakes UVC light 302 from light source 303 and channels the light in such a manner that it is evenly distributed 304 through the chamber.

FIG. 400 depicts an improved irradiation chamber with an expanded view 401 of the interior surface of said irradiation chamber. It is configured to use a tertiary optic 402 comprising a multi-faceted textured surface to diffuse light energy. Said tertiary optic intakes UVC light energy and diffuses it in a randomized homogeneous irradiance 403 and channels the light in such a manner that it even further distributes the light energy 403.

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of the present invention.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. A chamber for irradiating biological pathogens in fluids comprising:
a top and a bottom opposite the top, wherein the top and the bottom are substantially flat and substantially parallel to each other;
a first side and a second side opposite the first side, wherein the first side and the second side are substantially flat and substantially parallel to each other, and wherein the top, bottom, first side and second side form a substantially polygonal configuration;
a proximal face and a distal face opposite the proximal face enclosing the top, bottom, first side and second side forming an interior and an exterior of the UVC chamber, wherein the distal face comprises one or more substantially flat surfaces;
a first opening adjacent the proximal face, the opening configured to allow a fluid to flow into the interior of the chamber;
a second opening configured to allow the flow of a fluid from the interior of the chamber to the exterior of the UVC chamber;
a UVC light source positioned adjacent to the first opening of the UVC chamber; and wherein the interior of the UVC chamber comprises a plurality of facets configured at different angles to maximize internal reflection of UVC within the UVC chamber and the interior of the UVC chamber comprises reflective material configured to act as a light channeling optic to maximize internal reflection of UVC within the UVC chamber and to increase homogeneity and intensity of UVC radiation.

2. The chamber as recited in claim 1, wherein a secondary optic, comprising a transparent material using a convex lens, a diffuser, or some combination of a convex lens or diffuser, is attached to the first opening.

3. The chamber as described in claim 1, wherein UVC light entering the chamber is confined by means of reflection from the side walls that are made of materials that diffuse light; or
Or said walls are coated with materials that diffuse light.
